(19) United States
(12) Patent Application Publication
(10) Pub. No.: US 2009/0032031 A1
(43) Pub. Date: Feb. 5, 2009

(54) Special custom and ready made earplug devices for domestic animals

(76) Inventors: Viki E. Goyer, Muncie, IN (US); John W. Skidmore, Muncie, IN (US)

Correspondence Address: John D. Ritchison;RITCHISON LAW OFFICES, PC, Suite A, 115 East Ninth Street, Anderson, IN, 46016-1509, US

(21) Appl. No.: 12/221,488

(22) Filed: Aug. 4, 2008

Related U.S. Application Data
(60) Provisional application No. 60/963,266, filed on Aug. 4, 2007.

(51) Int. Cl. A61F 11/00; A61B 19/00; A42B 1/06

(52) U.S. Cl. 128/864; 128/898; 2/209

(57) ABSTRACT

A unique safety device used as an Earplug for Domestic Animals. Particularly this new Special earplug device for Pets is related to devices and methods to protect the ears during grooming from exposure to liquids and high sound levels. This Special earplug device for Pets is a new combination of existing materials configured with unique features that provide an effective, practical and economical way to protect the ear canals of animals.

This application claims the benefit of Provisional Patent Application Ser. No. 60/959,457 filed Aug. 4, 2007 by Viki E. Goyer and John W. Skidmore and entitled “Special Custom and Ready Made Earplug devices for Domestic Animals”.

### FIELD OF INVENTION

This invention relates to Special Custom and Ready Made Earplug devices for Domestic Animals. Particularly this new Special earplug device for Pets is related to safety devices and methods to protect the ears during grooming from exposure to liquids and high sound levels. This Special earplug device for Pets is a new combination of existing materials configured with unique features that provide an effective, practical and economical way to protect the ear canals of animals. The preferred and alternative embodiments are described below.

### FEDERALLY SPONSORED RESEARCH

NONE.

### SEQUENCE LISTING OR PROGRAM

NONE.

### BACKGROUND—FIELD OF INVENTION AND PRIOR ART

As far as known, there are no other Special Custom and Ready Made Earplug devices for Domestic Animals at the present time fully provide these improvements and functional characteristics as the present Special earplug devices Pets. It is believed that this device is made with fewer parts with improved configurations and physical features to provide more functionality when compared to other currently utilized ear protection means in the pet industry.

The particular simple combinations of materials and features are unique and novel and are not anticipated by prior art. Likewise, use of a Special earplug device for Pets provides significant benefits compared to prior art devices.

In the Pet Product Industry, statistics show that there are over 73.9 million dogs owned in 69.1 million households in the U.S. according to APPMA. These industry financial statistics show the pet industry as one of the fastest growing sub sectors in the entire U.S. economy, growing as much as 6% a year according to MSN Money “Investors Make the Most of a Dog's World”.

### A. Introduction of the Problems Addressed

Many of these pets are groomed by professional groomers. The animals are washed and rinsed. This grooming often uses high-speed dryers to quickly dry the fur of the animals. The dryers have high decibel sound levels. The unsafe exposure to water and high sound are not optimum for the pets. Often, groomers attempt to mitigate the water intrusion and sound exposure by placing cotton or other textile plugs in the ear canals. These are essentially ineffective for sound reduction and absorb the liquid. They can also become lodged in the ear canal presenting other health issues.

### B. Prior Art

The historical technology focused mainly on human earplugs and rarely tries to apply the technology to domestic animals. Examples of prior art devices begin with U.S. Pat. No. 2,070,403 issued to Hershman (1937) and entitled “ear fittings for excluding water or diminishing sound”. This invention teaches a molded plug for use with humans and mentions no use or application to animals for a safety device. No anticipation of universal basis with any nonhuman is taught. Another earplug device is taught by Draper, Jr. in U.S. Pat. No. 4,219,018 (1980) and entitled “Earplug unit with inserter and tie”. This teaches an Earplug unit with inserter and tie adapted to be worn as a unit which can be folded and compacted into a relatively small carrying case, comprises one or a pair of earplugs each having an outer end attached to one or both ends of a tie member on which is slideably carried and retained an earplug inserter moveable from a carrying position into engagement with the outer end of an earplug for its insertion into the auditory canal of an ear.

A further example of an earplug device that is consumable is shown by Castillo in U.S. Pat. No. 5,881,729 (1999) and entitled “Water sports ear plug”. This teaches a moldable device more complex than the Goyer invention and fails to teach use with animals. Another example of prior art in this field is a “Protective ear covering for animals” shown by Lowry in U.S. Pat. No. DES 417,315 (1999). The device a muff like design but has no detail as to function. It teaches a cover device not a plug device as Goyer demonstrates.

In the U.S. Pat. No. 6,310,961 (2001), Oliveira, et al. received a patent entitled “Disposable sleeve assembly for sound control device and container therefor”. It taught a simple and inexpensive sleeve assembly that is easy to apply to and remove from the distal end portion of a sound control device and can be adapted to provide circumferential contact with a user's ear canal. The sleeve assembly comprises a layer of flexible material but shows and teaches only human use.

A U.S. Patent Application Publication 20070062462 (2007) was submitted by Michele McGuire and is entitled “Animal noise protector”. This office action cited the above design patent U.S. Pat. No. DES 417,315 by Lowery as anticipating the McGuire device.

### SUMMARY OF THE INVENTION

A Special Custom and Ready Made Earplug devices for Domestic Animals have been developed and designed to provide a unique combination that is specifically related to devices and methods to protect the ear openings (canals) of pets during grooming. The Special safety earplug device for Pets may be used with dogs, cats, and other animals that are groomed with liquids and/or exposed to dryer devices. The new devices present significant ear protection for the animals. The devices are made of high quality, durable materials. The materials and configurations are pet safe and non-toxic to animals and humans.

The preferred embodiment of the Special Custom Earplug devices for Domestic Animals is comprised of a custom made plug device of a soft urethane or the like that has been custom molded from a specific molded impression of the animal's ear canal. The mold impression is then used to produce a custom ear plug configured to the shape and size of the specific animal's ear canal. The alternative embodiment is a ready-made cylindrical-like urethane or the like in various thicknesses that is presented in lengths of tubular-like foam that may be cut to various lengths and then placed into the animal's ear canal without the need of pre-impression molds and wait times for a custom made ear plug.

### OBJECTS AND ADVANTAGES

There are several objects and advantages of the Special Custom and Ready Made Earplug devices for Domestic Animals. There are currently no known ear protection devices that are effective at providing the objects of this invention.

The following TABLE A summarizes various advantages and objects of the Special Custom and Ready Made Earplug devices for Domestic Animals. This list is exemplary and not limiting to the many advantages offered by this new device.

TABLE A
Various Benefits, Advantages and Objects
This device:

| ITEM | BENEFIT |
|---|---|
| 1. | Reduces water intrusion to the pet during “grooming”. |
| 2. | Reduces infection and complications from water |
| | intrusion during grooming and swimming |
| 3. | Reduces exposure to high decibel sound levels of hair |
| | dryers and the like used on pets and animals during |
| | the grooming process. Helps reduce long-term exposure |
| | to high decibel exposure from drying devices used |
| | during grooming. |
| 4. | Provides an easy device and method to insert and |
| | remove sound reduction devices. |
| 5. | Is versatile to various sizes (Small, Medium, and |
| | Large). |
| 6. | Is a drug free control of an animal's exposure to |
| | noise (gun shots, blasts, etc. |
| 7. | May be with a cord retrieval or other means to remove |
| | from the ear canal of the animal |

Noteworthy to an observer is that other advantages and additional features of the Special Custom and Ready Made Earplug devices for Domestic Animals will be more apparent from the accompanying drawings and from the full description of the device. For one skilled in the art of pet and animal protection devices—especially for ears—it is readily understood that the features shown in the examples with this device is readily adapted for improvement to other types of mechanisms and devices for use with pets and other animals.

### DESCRIPTION OF THE DRAWINGS—FIGURES

The accompanying drawings, which are incorporated in and constitute a part of this specification, illustrate a preferred and alternative embodiments for the Special Custom and Ready Made Earplug devices for Domestic Animals. The drawings together with the summary description given above and a detailed description given below serve to explain the principles of the Special Custom and Ready Made Earplug devices for Domestic Animals. It is understood, however, that the device is not limited to only the precise arrangements and instrumentalities shown.

FIGS. 1A through 1E are sketches that depict the general preferred embodiment for the Special Ready Made Earplug devices for Domestic Animals.

FIG. 2A through 2D are sketches that show the ready-made version of the device for providing and Earplug device for Domestic Animals.

FIGS. 3A through 3D are sketches that show more of the ready-made Earplug devices for Domestic Animals.

FIGS. 4A and 4B are additional sketches that show details and features of the preferred embodiment on an animal.

FIGS. 5A through 5C are sketches that show a simple one-piece Earplug devices for Domestic Animals with similar configurations.

FIGS. 6A through 6C are sketches that show details and features of the preferred embodiment of the Special Custom Earplug devices for Domestic Animals and an animal's ear canal and ear physiology.

FIGS. 7A through 7C are sketches of a two-part mixture to create a mold for producing a custom made Earplug devices for Domestic Animals.

### DESCRIPTION OF THE DRAWINGS—REFERENCE NUMERALS

The following list refers to the drawings.

| Ref # | Description |
|---|---|
| 30 | Animal such as a dog, cat, or the like |
| 30A | Ear of an animal 30 |
| 31 | General and alternative embodiment of the ready made earplugs |
| 31A | End surface of the ready made earplug |
| 31B | Side surface of the ready made earplug |
| 31C | Contoured tip of the ready made earplug |
| 31D | Flared end of the ready made earplug |
| 31E | General contoured type of a ready made earplug |
| 32 | Means to retain earplug or attach to another plug or object such |
| | as a cord, string, lanyard, wire or the like |
| 33 | Loop to secure to an object such as a collar |
| 34 | Position “behind an ear” 30A |
| 40 | Coil of earplug material |
| 41 | Alternative embodiment of a ready made earplug with a cross |
| | section such as a circular, hexagon, oval or other polygon-like |
| | area |
| 41D | Variable essential diameter of a cross section of earplug 41 |
| 42 | Dispenser device |
| 43 | Earplug cutting means |
| 44 | Dispenser point |
| 51 | Alternative embodiment of a ready made earplug with a round |
| | cross section and flat end at each end of the plug |
| 52 | Alternative embodiment of a ready made earplug with a round |
| | cross section, one flat end and one expanded cap at the opposite |
| | end of the plug |
| 53 | Alternative embodiment of a ready made earplug with a round |
| | cross section, one flat end and one expanded cap at the opposite |
| | end of the plug whereby the sides are tapered |
| 60 | Audiography sketch of the ear of an animal |
| 61 | Ear canal |
| 62 | Preferred embodiment of molded Impression material |
| 62A | Impression material |
| 63 | Cotton mass (ball or the like) |
| 64 | Retention cord or string attached to the cotton |
| 70 | Part A of impression material |
| 71 | Part B of impression material |
| 72 | Application means such as a syringe or the like |
| 73 | Partial mixture of the impression material |
| 74 | Mixing means such as a stick or paddle |

### DETAILED DESCRIPTION OF PREFERRED EMBODIMENT

The present invention presented is a Special Custom Earplug devices for Domestic Animals 62. Particularly the preferred embodiment of the device is related to devices and methods that provide a unique combination which has been specifically configured to provide ear protection for animals during grooming, and the like. The preferred embodiment of the Special Custom Earplug devices for Domestic Animals 62 is comprised of a custom made plug device of a soft urethane or the like that has been custom molded from a specific molded impression of the animal's ear canal. The mold impression is then used to produce a custom ear plug 62A configured to the shape and size of the specific animal's ear canal. An alternative embodiment 31 is a ready-made cylindrical-like urethane or the like in various thicknesses or diameters that is presented in lengths of tubular-like foam that may be cut to various lengths and then placed into the animal's ear canal without the need of pre-impression molds and wait times for use as a ready-made ear plug. Other alternative embodiments 41, 51, 52, and 53 include different cross sections of material shaped in various configurations as described below. Each of these earplugs may be individual units or paired together and secured by a cord 42. All these embodiments provide a unique departure and innovation to protecting domesticated animals during the grooming process.

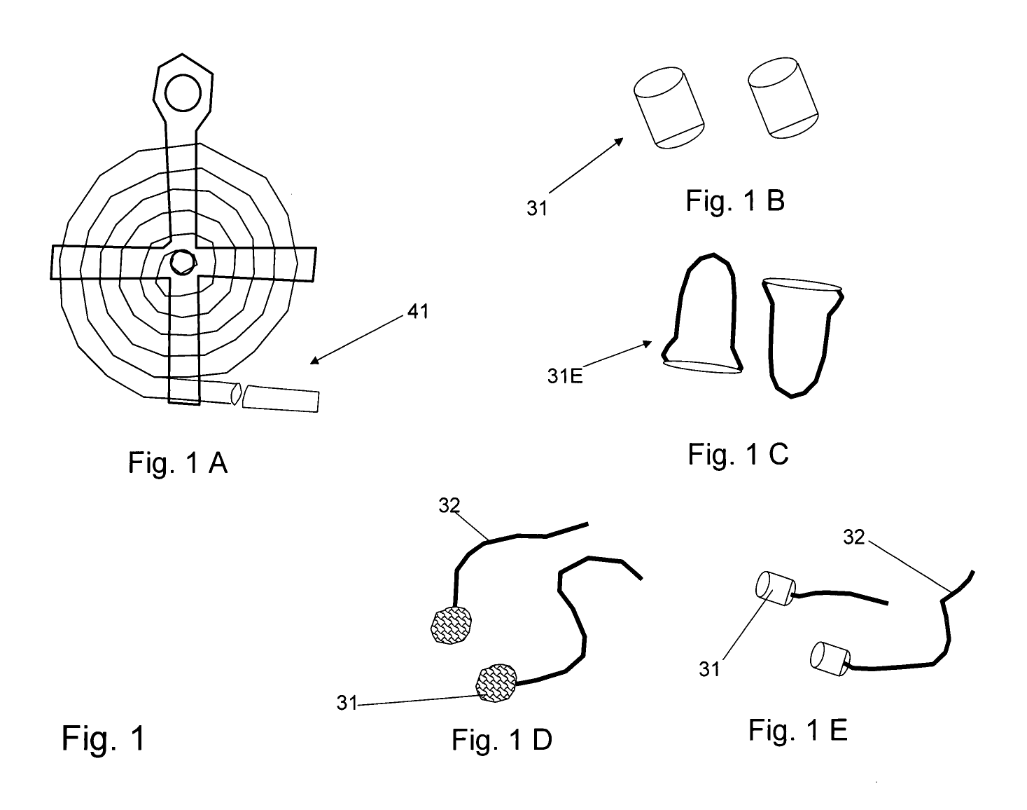

There is shown in FIGS. 1-7 a complete detail and operative embodiment of the Special Custom and Ready Made Earplug devices for Domestic Animals 31, 41. In the drawings and illustrations, one notes well that the FIGS. 1-7 detail the special configuration and method(s) of operation for the Special Custom and Ready Made Earplug devices for Domestic Animals 31.

The advantages for the Special Custom made and ready made Earplug devices for Domestic Animals 31, 41 are listed above in the introduction. Succinctly the benefits are the device:
Reduces water intrusion to the pet during “grooming”.
Reduces infection and complications from water intrusion during grooming and swimming
Reduces exposure to high decibel sound levels of hair dryers and the like used on pets and animals during the grooming process.
Helps reduce long-term exposure to high decibel exposure from drying devices used during grooming.
Provides an easy device and method to insert and remove sound reduction devices.
Is versatile to various sizes (Small, Medium, and Large).
Is a drug free control of an animal's exposure to noise (gun shots, blasts, etc.
May be with a cord retrieval or other means to remove from the ear canal of the animal

The preferred embodiment of the Special Custom Earplug devices for Domestic Animals 62 is comprised of a custom made plug device of a soft urethane or the like that has been custom molded from a specific molded impression material 62A of the animal's ear canal. The mold impression 62A is then used to produce a custom ear plug 62 configured to the shape and size of the specific animal's ear canal 61. The alternative embodiment 41 is a ready-made cylindrical-like urethane or the like in various thicknesses that is presented in lengths of tubular-like foam that may be cut to various lengths and then placed into the animal's ear canal 61 without the need of pre-impression molds and wait times for a custom made ear plug 62.

The accompanying drawings, which are incorporated in and constitute a part of this specification, illustrate a preferred embodiment of the Special Custom Earplug device 62 or alternative ready made devices 31, 41, 51, 52, and 53 for Domestic Animals and Pets. The drawings together with the summary description given above and a detailed description given below serve to explain the principles of the Special Earplug devices for Domestic Animals 62, 31, 41, 51, 52, and 53. It is understood, however, that the preferred and alternative devices 62, 31 et al are not limited to only the precise arrangements and instrumentalities shown.

FIGS. 1A through 1E are sketches that depict the general preferred embodiment for the Ready Made Earplug devices 31, 41 et al for Domestic Animals 30. In FIG. 1A, the general “cut-to-length” plug 41 is shown. FIGS. 1B and 1 C show pre-formed devices 31, 31E and FIGS. 1D and 1E show devices 31 with retention cords 32 to couple and secure the devices. These are described further in the below details.

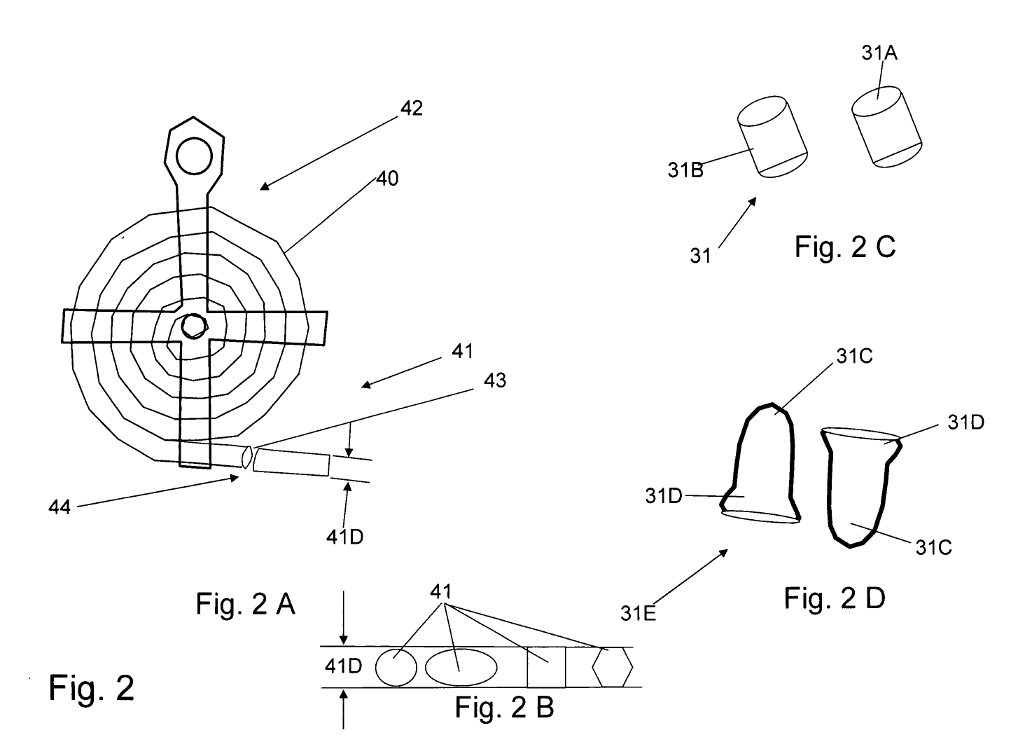

FIG. 2A through 2D are sketches that show the Ready Made Earplug devices 31, 41 et al for Domestic Animals 30. FIG. 2A demonstrates a “cut-to-length” device 41 that is distributed from a dispenser 42. The coil of material 40 is presented to a dispensing point 44 and the cut to length by a cut-off means 43 such as a knife or automatic slicing means. FIG. 2B reinforces that the roll 40 and earplug 41 may have variable diameters 41D and different geometrical cross sections, such as (for example and not limitation) a circle, ellipse, square, hexagon and the like. The materials used may be of several types. A foam, urethane, composite materials, or other available options that are suitable for ear canal exposure are anticipated. A rating of R22 minimum is required for sound protection and may be made from the various silicone, foam, beeswax or other composite material that are suitable. FIGS. 2C and 2D show discrete earplugs 31, 31E. The regular cylindrical types 31 normally have end surfaces 31A and side surfaces 31B. The more elaborate contoured plugs 31E have a contoured end 31C and a flat, expanded end 31D. The materials are similar as for the cut-to -length devices 41 described above. Sizes may vary for use with animals 30 that have variable sized ear canals 61.

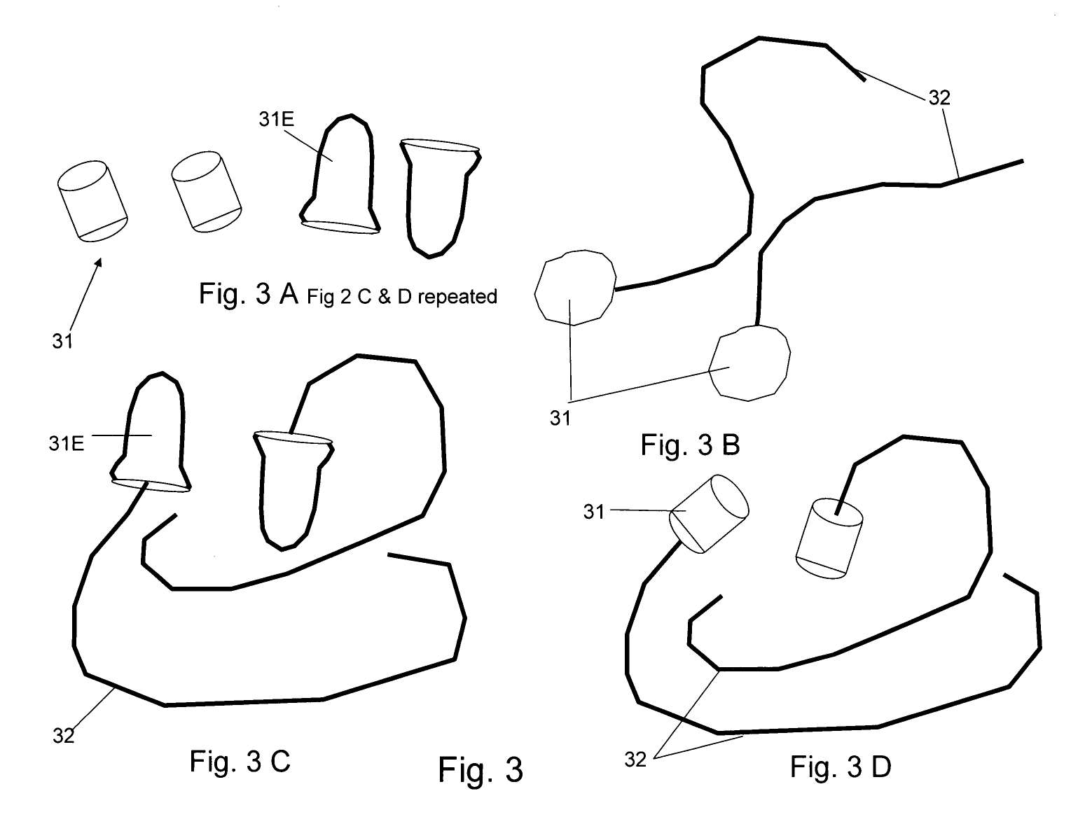

FIGS. 3A through 3D are sketches that show more of the ready-made Earplug devices 31 for Domestic Animals. Here the various sketches reinforce the ability to use different configurations as single devices and with or without retention means 32. Important to note is that some similar devices have been used for sound barrier plugs for humans but do not teach the new use of sound barriers for animals or water intrusion preventers for animals. This new use is novel to the Special Custom 62 and Ready Made Earplug devices 31, 41 et al for Domestic Animals and is not anticipated or claimed in prior art.

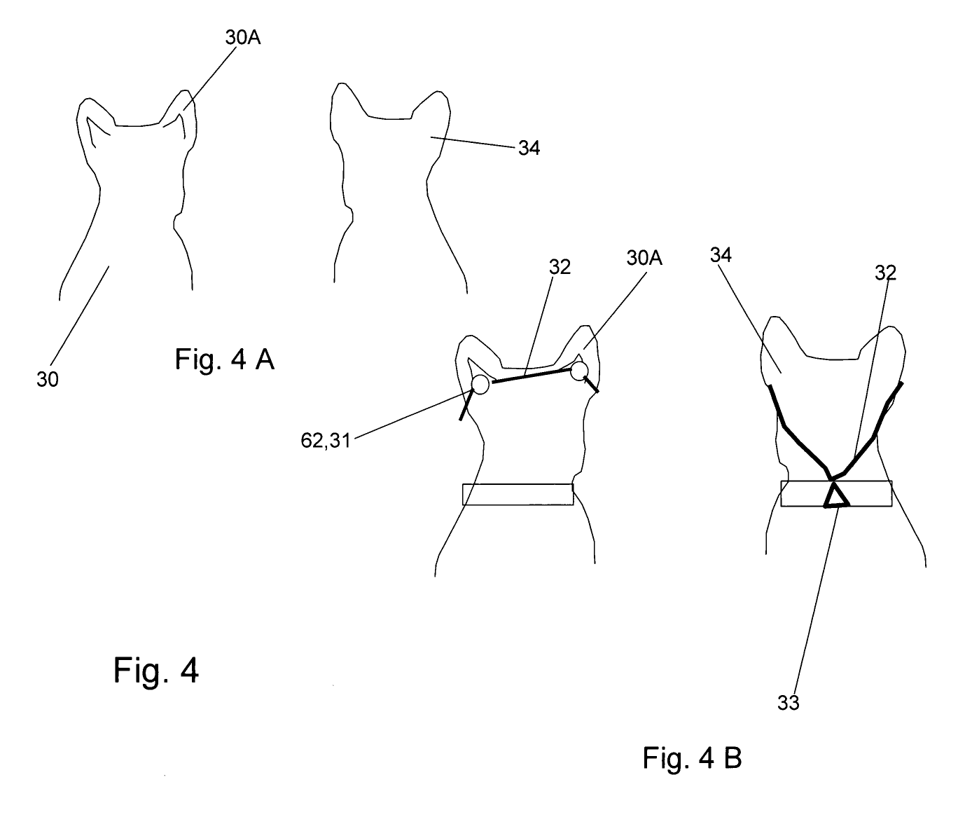

FIGS. 4A and 4B are additional sketches that show details and features of the preferred embodiment on an animal 30. FIG. 4A depicts an animal 30 with ears 30A and a position behind its ears 34. FIG. 4B shows an earplug device 62, 31 which has the plugs connected by means of a cord 32 or lanyard, etc. This cord 32 is directed or placed behind the ear 34 and may also be attached to an object such as a collar by an attachment means or loop 33. This keeps the earplugs 62, 31 visible to the owner or groomer and prevents leaving the plugs 62, 31 in the animal 30 after the grooming, exposure to water, or exposure to sound. A flexible, rigid or semi rigid cord 32 may be attached from the right to the left ear device 31, 41, 62, et al.

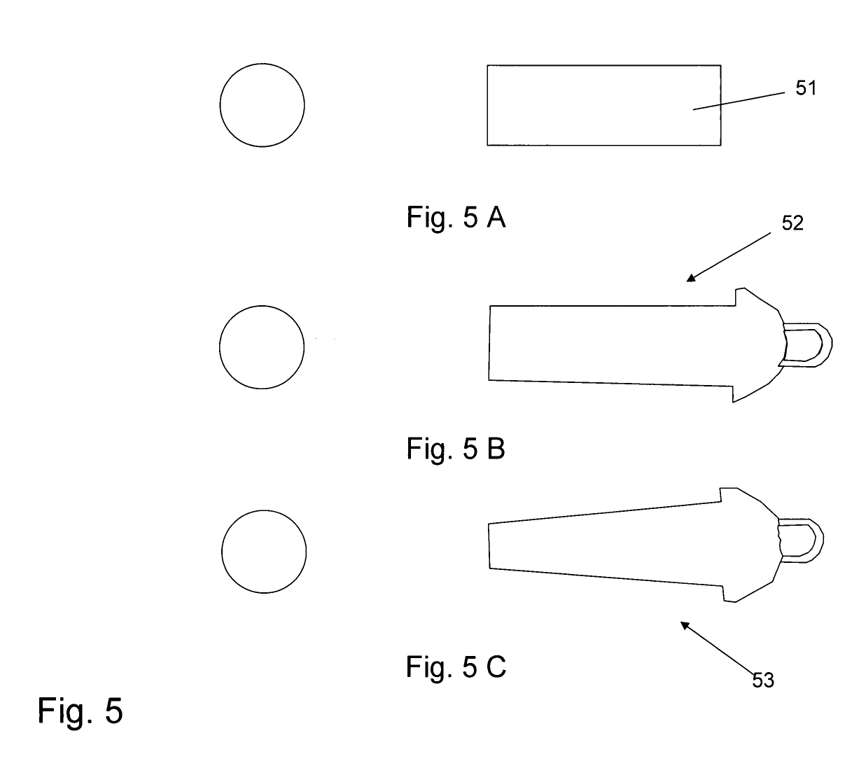

FIGS. 5A through 5C are sketches that show a simple one-piece Earplug devices 51, 52, 53 for Domestic Animals with similar configurations. The Figs. are self explanatory. One Alternative embodiment 51 shows a ready made earplug with a round cross section and flat end at each end of the plug. Another Alternative embodiment 52 shows a ready made earplug with a round cross section, one flat end and one expanded cap at the opposite end of the plug. The final discrete Alternative embodiment 53 shows a ready made earplug with a round cross section, one flat end and one expanded cap at the opposite end of the plug whereby the sides are tapered.

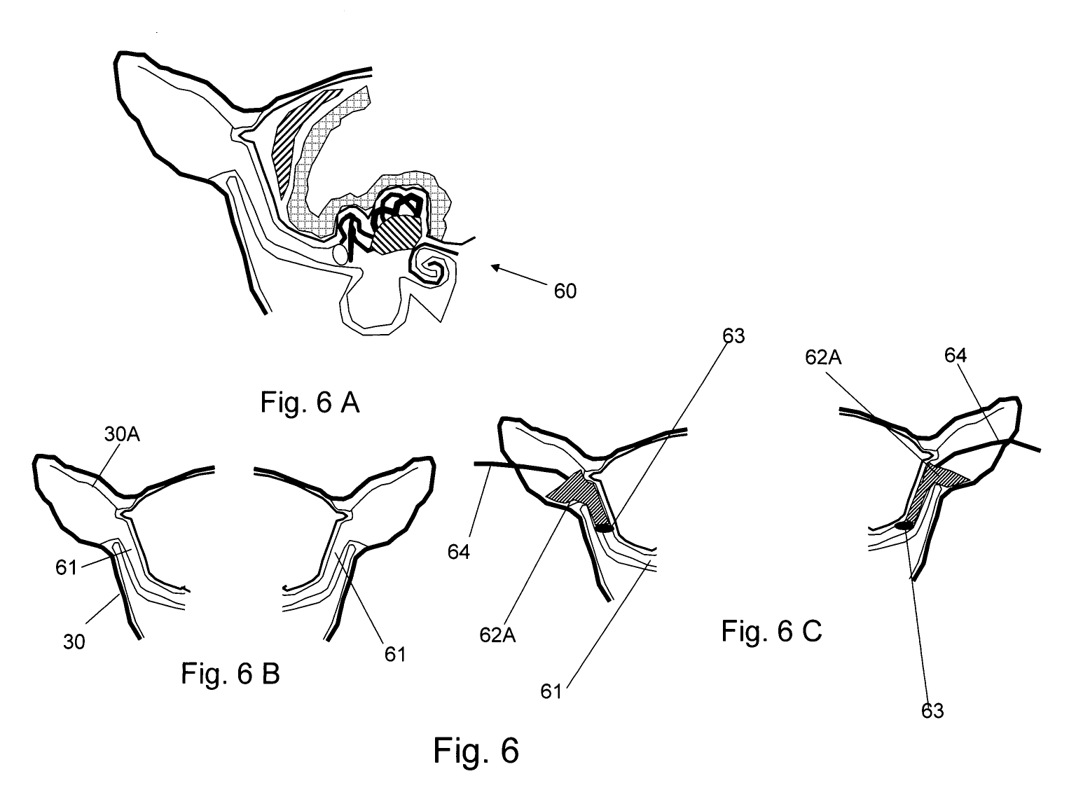

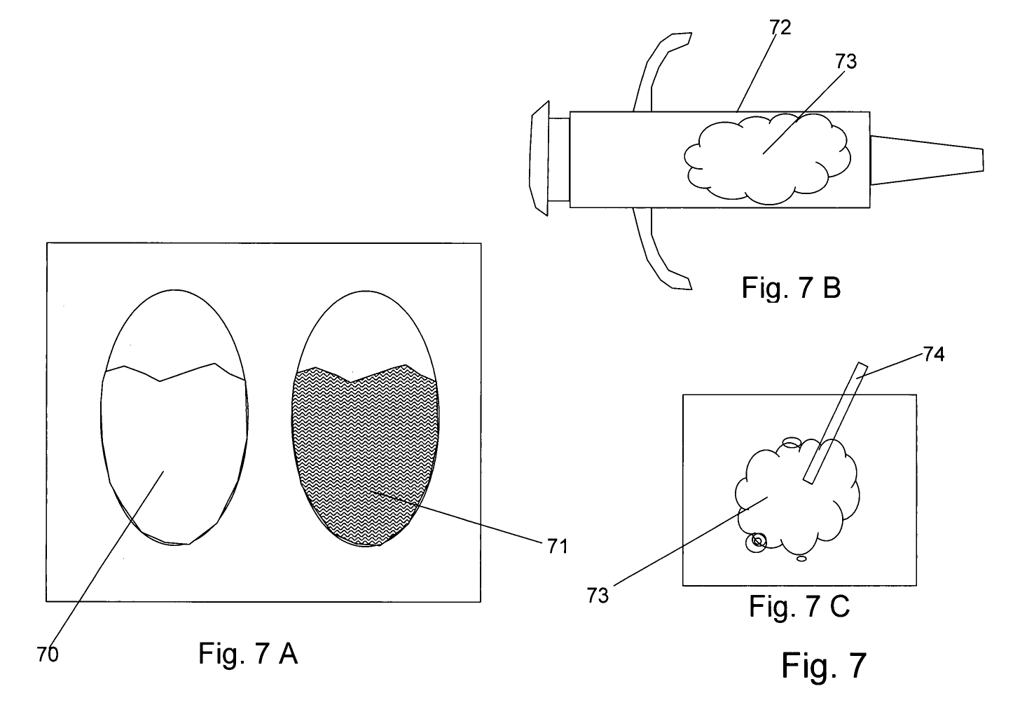

FIGS. 6A through 6C are sketches that show details and features of the preferred embodiment of the Special Custom Earplug devices 62 for Domestic Animals and an animal's ear canal 61 and ear physiology. FIG. 6A shows an audiography sketch of an animals ear 30A and ear canal 61. The various parts of the ear 30A are demonstrated. Special attention is directed to the ear canal 61. FIG. 6B shows a similar sketch showing the animal 30, the ear 30A of the animal, and the ear canal 61. FIG. 6C shows the main features of the Special Custom Earplug devices 62 for Domestic Animals. Here the cotton damming 63 with a retention string or cord 64 is shown. The impression material 62A in the canal 61 is also shown. How these are used is shown below in the Operation Description. What the materials, mix and application are described in FIG. 7.

FIGS. 7A through 7C are sketches of a two-part mixture to create a mold for producing a custom made Earplug devices 62 for Domestic Animals. It is recommended that the custom ear mold impressions should be made by a Veterinarian. The operation and method of use is described below. The mold material will depend on the final use of the mold, either as a water barrier, or for sound protection or both. A rating of R22 minimum is required for sound protection and may be made from silicone, foam, beeswax or other composite material that is suitable for the intended purpose of the mold. Materials used are of the type used to make dental impressions and are non Allergenic and hygienic. Materials that may be used include Vinyl Polysiloxane, Silicone, or other composite material or the like suitable for obtaining an impression. The material for the ear canal dam 63 may be cotton, foam or any suitable non allergenic hygienic material. FIG. 7A shows a two part mixture 70, 71. These are maintained in airtight, watertight packages until use. The Part A 70 and Part B 71 are placed together and mixed by a means to produce the composition and mixture 73 which eventually sets-up or hardens to 62A. While this is pliable, the mixture 73 is placed in an application means 72 such as a syringe or the like to facilitate placing the mixture 73 into the ear canal 63 of the animal 30.

All of the details mentioned here are exemplary and not limiting. Other components specific to describing the Special Custom 62 and Ready Made Earplug devices 31, 41 et al for Domestic Animals may be added as a person having ordinary skill in the field of pet and ear protective devices well appreciates.

### Operation of the Preferred Embodiment

The preferred and alternative embodiments for the Special Custom 62 and Ready Made Earplug devices 31, 41 et al for Domestic Animals have been described in the above paragraphs. The manner of how the device operates is described below. One skilled in the art of pet protective devices will note that the description above and the operation described here must be taken together to fully illustrate the concept of the Special Custom 62 and Ready Made Earplug devices 31, 41 et al for Domestic Animals.

The preferred embodiment of the Special Custom Earplug devices 62 is comprised of a molded earplug 62 that has originated from a custom made impression of the ear canal 61 of an animal with a special impression material 62A. FIGS. 6 and 7 show parts of the process by sketches. The impression material 62A is prepared by mixing Part A 70 and Part B 71 and thereby forming a partial mixture 73. This mixture 73 is placed in an application means such as a syringe 72. The animal 30 is prepared by placing a cotton ball 63 with a retention cord 64 into the ear canal 61. The material 73, 62A is placed in the canal 61. The material 62A is allowed to harden or set-up. The cotton ball 63 is then pulled out of the canal 61 by the cord 64. The impression material 62A is then used as a pattern for making the “permanent” earplug 62.

It is recommended that the custom ear mold 62A impressions should be made by a Veterinarian. The custom molds 62A are a specific size for that dog 30 only and would be used only by that dog. A cotton or foam dam 63 with a length of string 64 attached, is inserted into the ear canal of the dog by using a hemostat or tweezers. The dam is placed at the bottom of the ear canal 61 and should not be forced any further. The dam is to protect the ear drum from the impression material. The impression material 62A is then mixed according to package directions and then placed in a large syringe 72. The material 73 will be slightly less dense than putty, but not liquid. The impression material 73, 62A is delivered into the ear canal 61 of the dogs 30 ear 30A. The time for the material to set will vary from one to five minutes. The “hardened” impression material 62A is then removed from the dog's ear 30A. The dam protector 63 is removed by pulling the string 64 to remove it from the ear canal 61. The impression 62A is then sent to a manufacturer to make a permanent mold 62.

This method or procedure is summarized as the following table:

| Step | Description |
|---|---|
| 1 | Plug Ear canal 61 of the animal 30 with cotton mass 63 |
| | with retention means 64 or equal |
| 2 | Mix mold material 70, 71 |
| 3 | Inject/place mold material 73 in ear canal 61 |
| 4 | Remove molded form 62A from canal 61 by gently tugging |
| | retention cord 64 and cotton ball 63 |
| 5 | Use molded form and mold for actual custom device 62 |
| 6 | Make custom device 62 |
| 7 | Confirm fit in animal 30 and its ear canal 61 |
| 8 | Use as sound reducing or liquid intrusion reducing |
| | device 62 with the animal 30 |

The other alternative embodiments and Ready Made Earplug devices 31, 41 et al for Domestic Animals are used with the animal but requires less preparation. The ready made embodiments that are preformed earplugs such as described above Ready Made Earplug devices 41, 51, 52, and 53 are ready for immediate use. One merely needs to determine which thickness or diameter 41D of the earplug best fits the animal's 30 ear canal 61. Once selected, the earplug is placed in the animal for bathing, swimming, travel or other uses.

The various uses for a Special Custom 62 and Ready Made Earplug devices 31, 41 et al for Domestic Animals et al for Domestic Animals are shown in the following table:

| Item | Description |
|---|---|
| 1 | Grooming of animals for water intrusion and sound |
| | reduction |
| 2 | Training of animals for sight not sound |
| 3 | Training of animals around gunshot or explosive blasts |
| 4 | Training of animals in and around water |
| 5 | Training and transport of animals in air travel |
| 6 | Other Behavior training |

The one ready made plug 41 may require additional preparation prior to use. The designs will allow for choices of several design cross sections (FIG. 2B) s as well as colors. Some designs may be a combination of sizes, utilizing a “cut to fit” style for a particular size that is needed. This cut to fit plug 41 comes on a coil of material 40 and is placed on a dispenser device 42. The amount or length is pulled past a dispenser point 44 and then sliced or cut to a desired length by a cutting means 43 such as a knife, scissors or built-in cut-off means (not shown, but understood and well appreciated by one skilled in the art of cutting devices). The cut-to-length earplug 41 is then placed in the ear canal 61 of the animal 30.

For the custom and ready made devices, a short training video and training pamphlet may be included with the product to help the pet owner train their dog to accept and wear the earplugs.

With the above description it is to be understood that the Special Custom 62 and Ready Made Earplug devices 31, 41 et al for Domestic Animals is not to be limited to only the disclosed embodiments. The features of the Special Custom 62 and Ready Made Earplug devices 31, 41 et al for Domestic Animals are intended to cover various modifications and equivalent arrangements included within the spirit and scope of the description.

What is claimed is:

1. A ready made earplug device for use with a Domestic Animal made of a pliable material and configured to be firmly and tightly placed within an ear canal of the animal
wherein the device may be used to prevent air, sound, and water intrusion into an ear canal of the animal during a grooming process and swimming

2. The device according to claim 1 wherein the pliable material is silicone.

3. The device according to claim 1 wherein the pliable material is foam.

4. The device according to claim 1 wherein the pliable material is beeswax.

5. The device according to claim 1 wherein the pliable material is a composite material.

6. The device according to claim 1 wherein the pliable material is non Allergenic and hygienic.

7. The device according to claim 1 wherein the configuration is a regular cylindrical types cut to length. 1 normally have end surfaces 31A and side surfaces 31B. The. The materials are similar as for the cut-to -length devices 41 material is silicone.

8. The device according to claim 1 wherein the configuration is a more elaborate contoured plugs 31E have a contoured end 31C and a flat, expanded end 31D.

9. A special custom made Earplug device for use with a Domestic Animal made of a pliable material and configured to be firmly and tightly placed within an ear canal of the animal wherein the device may be used to prevent air, sound, and water intrusion into an ear canal of the animal during a grooming process and swimming

10. A method for forming a Special Custom Made Earplug devices for Domestic Animals devices comprising:
a) STEP 1: Plug Ear canal 61 of the animal 30 with cotton mass 63 with retention means 64 or equal;
b) STEP 2: Mix molding material 70,71;
c) STEP 3: Inject/place mold material 73 in ear canal 61;
d) STEP 4: Remove molded form 62A from canal 61 by gently tugging retention cord 64 and cotton ball 63;
e) STEP 5: Use molded form and mold for actual custom device 62;
f) STEP 6: Make custom device 62;
g) STEP 7: Confirm fit in animal 30 and its ear canal 61; and
h) STEP 8: Use as sound reducing or liquid intrusion reducing device 62 with the animal 30.
